(19) United States
(12) Patent Application Publication
(10) Pub. No.: US 2021/0196171 A1
(43) Pub. Date: Jul. 1, 2021

(54) WEARABLE APPARATUS FOR SENSING STRESS AND METHOD OF USE THEREOF

(71) Applicants: King Abdullah University of Science and Technology, Thuwal (SA); Swansea University, Swansea (GB)

(72) Inventors: Carlos M. Duarte, Thuwal (SA); Rory Wilson, Swansea (GB)

(21) Appl. No.: 17/058,385

(22) PCT Filed: May 27, 2019
(86) PCT No.: PCT/IB2019/054385

§ 371 Date: Nov. 24, 2020

Related U.S. Application Data
(60) Provisional application No. 62/676,808, filed on May 25, 2018.

(51) Int. Cl. A61B 5/16; A61B 5/08; A61B 5/11; A61B 5/00

(52) U.S. Cl. CPC A61B 5/165; A61B 5/0816; A61B 5/1118; A61B 5/742; A61B 5/6823; A61B 2562/0261; A61B 5/0002; A61B 2562/0219; A61B 2562/0223; A61B 2503/40; A61B 5/6831

(57) ABSTRACT

Wearable devices that track activity and ventilation rates for use on humans or animals that breathe air and methods of use thereof, are provided. The device is generally suitable to be worn around a subject's chest, and typically contains one or more sensors. Generally, one sensor detects respiratory rate and another sensor detects acceleration (i.e. activity). The method and apparatus can be used to determine the minimum ventilation rate for a given degree (intensity) of movement to construct the ‘stress-free’ relationship between ventilation rate and movement. Then measured values of ventilation rate in relation to movement are compared to the predicted, minimum ventilation rates (stress-free values) and the minimum ‘stress-free’ values are subtracted from the measured values. The difference (Ventilation Rate Above Predicted’ (VRAP)) correlates with stress in the subject.

### CROSS-REFERENCE TO RELATED APPLICATIONS

This application claims the benefit of and priority to U.S. Provisional Application Ser. No. 62/676,808, entitled Stress Sensor And Metric Based On Ventilation Rate Above Predicted, filed May 25, 2018, the entire contents of which is incorporated by reference in its entirety.

### FIELD OF THE INVENTION

The invention generally relates to apparatus and methods for evaluating physiological properties in a human or animal, particularly those relating to stress responses.

### BACKGROUND OF THE INVENTION

Oxygen use in people, and animals, increases with activity. As a result, ventilation, or breathing rate, also increases with activity as the body takes measures to enhance oxygen supply to the blood. Breathing rate quickens when people are stressed. Thus, at any one time, the body has a breathing rate that is composed of a proportion that contributes to oxygenation due to exercise and a proportion that is due to stress. However, what portion of the breathing rate is related to a stress response is generally unknown.

There is a need for devices and methods that are able to measure one or more physiological properties that can change in response to stress and accurately determine the level of stress in a person or animal.

### SUMMARY OF THE INVENTION

Wearable devices that track activity and ventilation rates for use on humans or animals that breathe air, such as mammals and birds, and methods associated with using the same, are provided. The wearable device is generally suitable to be worn around a subject's chest (e.g., a thoracic strap). The device typically contains one or more sensors, preferably two sensors for monitoring one or more physiological parameters and movement by the individual or animal wearing the device. Generally, one sensor detects respiratory rate (i.e. ventilation rate) and another sensor detects acceleration.

The method and apparatus described herein can be used to determine the minimum ventilation rate for a given degree (intensity) of movement to construct the ‘stress-free’ relationship between ventilation rate and movement. Then measured values of ventilation rate in relation to movement are compared to the predicted, minimum ventilation rates (stress-free values) and the minimum “stress-free” values are subtracted from the measured values. The difference (“Ventilation Rate Above Predicted” (“VRAP”)) correlates with stress in the subject.

### BRIEF DESCRIPTION OF THE DRAWINGS

FIG. 1A is a series of spherical plots (“urchin” plots) showing changes in activity of a wild-caught, sedated, and released European badger over the first 6 days post-release. FIG. 1B is a graph showing vectorial sum of the dynamic body acceleration (VeDBA) values over time (days) and shows generally reduced VeDBA activities initially, followed by increasing VeDBA values over time. The urchin plots in FIG. 1A represent 3 days within this period to provide more information on activity.

FIG. 2 is a graph showing ventilation rates as amplitude (gauss, G) over time (s) of a subject as measured with a magnet-driven magnetometer. The trace shows the raw signal in relation to the breathing. For this exercise, the participant was asked to lie and then execute three types of breathing patterns; SS1 & SS2—Short, sharp breaths, NN1 & NN2—Normal breaths, and FD—Full, deep breaths. λ indicates the wavelength (and hence the ventilation rate). Independent equipment was used to confirm that all breaths represented by the system corresponded to real breaths.

FIG. 3 is a graph showing the relationship between the tidal volume (L) and amplitude (G). The shaded area shows 95% confidence limits.

FIG. 4 is a graph showing changes in magnetic field strength (mV) and its corresponding VeDBA (g) over time (s). The example here is from a Magellanic penguin, placed in a darkened box. Even though there is no change in activity (as indicated by the VeDBA), the ventilation rate following stress at time 0 decreased. The VeDBA values (ca.0.3 g) indicate that the animal is predominantly “still” whereas, for comparison, the VeDBA for a walking penguin with an upper back-mounted tag are approximately 1.4 g.

FIG. 5 is a graph showing changes in ventilation rate (breaths per minute) and its corresponding VeDBA (g) over time (s) with changes in activity. Change in breathing rate according to activity of a person engaged in defined periods (each lasting for 1 minute) of sitting (white panels), standing (light shaded panels) and walking (shaded panels) is shown. The VeDBA is shown to indicate the level of activities and only shows a spike between sitting and standing, the subject otherwise being motionless (even ostensibly completely motionless people have some residual movement, which is why the VeDBA is not 0 at this time). As shown in FIG. 5, the breath rate uncouples from activity in anticipation of activity to come some 30 s before the new activity is undertaken.

FIG. 6 is a graph showing changes in breathing rate (breaths per minute) and its corresponding VeDBA (g) over time (s) when the subject is exposed to different stimuli. Change in breathing rate according to exposure to different stimuli for a sitting person is presented. The second panel from the left shows when the person was exposed to relaxing music (Toto; ‘Africa’ and Rusted Root; ‘Send me on my way’), the third panel shows when exposed to loud music (Aerosmith; ‘Don't want to miss a thing’) and the fourth panel shows when the person watched a horror sequence (a baboon eating a live impala).

FIGS. 7A and 7B are graphs showing changes in breathing rate (breaths per minute) over speed (km/h) when the person is moving on a treadmill (FIG. 7A), or at different VeDBA (g) corresponding to those speeds (FIG. 7B). The squares show the stabilized breathing rates (with SD error bars), while the circles show the breathing rates for the same conditions but with the person listening to loud music.

FIG. 8 is a diagram showing an exemplary stress sensor 10 with its elements: a magnet 20, a sensing apparatus 30 positioned on a thoracic strap 40 and transmission of VRAP signal 50 to an output display 60. The stress sensor includes (i) a breathing sensor and (ii) a movement sensor (both contained within or close to the sensing apparatus 30). The VRAP signal (a metric for stress) is displayed, and optionally stored, on an output device (not shown in Figure), such as a computer, watch or smart phone.

FIG. 9 is a graph showing a relationship between breathing rate (ventilation rate) in a human and activity, showing the minimum ventilation rate for any given level of activity (in small circles) and ventilation rates that do not correspond to this (big circles). The difference in ventilation rate between the grey circles and the minimum ventilation rate line (shown by different length arrows) is the ventilation rate above predicted (VRAP) and is a measure of stress.

### DETAILED DESCRIPTION OF THE INVENTION

### I. Definitions

The singular forms “a,” “an,” and “the” include plural reference unless the context clearly dictates otherwise. For example, reference to “a compound” includes a plurality of compounds and reference to “the compound” is a reference to one or more compounds and equivalents thereof known to those skilled in the art.

The terms “can,” and “can be,” and related terms are intended to convey that the subject matter involved is optional (that is, the subject matter is present in some forms and is not present in other forms), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present and instances where it does not occur or is not present.

As used herein, the term “subject” refers to humans and animals that breathe air.

As used herein, the term “garment” refers to any item that is adapted to cover at least a portion of a subject's body, including but not limited to, scuba wet suit, shirt, vest, jacket, band, strap, belt, and the like.

As used herein, the term “electrical communication” refers to communication in which any type of information, e.g. data, is transmitted by electric signals propagated over wires or wireless such as radio signals, e.g. bluetooth.

### II. Apparatus

Wearable devices that track activity level and ventilation rates for use on a subject that breathes air. The subject can be humans or animals that breathe air, such as mammals and birds, and methods associated with using the same, are provided.

The wearable device is generally suitable to be directly attached to a subject or embedded in or carried by a support structure, such as a monitoring garment that is suitable to be worn by the subject. The monitoring garment can include various garments or items that are adapted to tightly conform to at least a portion of a subject's body, such as a shirt, vest, jacket, band, strap, belt, and the like. In some instances, the support structure is a thoracic strap around the subject's chest, a belt around the subject's abdomen (e.g. a belt), or a combination thereof.

The device typically contains one or more sensors, preferably two sensors, and optionally more than two sensors, for monitoring one or more physiological parameters and movement by the individual or animal wearing the device. Generally, one sensor detects respiratory rate and another sensor detects acceleration. In some instances, the device includes more than one ventilation sensors and/or more than one activity sensors. Preferably, the ventilation sensors and activity sensors are on the same support structure. Optionally, the device includes additional sensors to monitor one or more additional physiological parameters, such as electrocardiogram, temperature, blood oxygen level, heart rate, pulse rate, blood pressure, etc.

The wearable devices can further includes a processor for processing the data from the sensors. The sensors can be in electrical communication with a processor that performs mathematical analysis using an appropriate algorithm or signal processing on the data information provided by the sensors and calculates the ventilation rate and processes the acceleration data. The electrical communication between the sensors and the processor can be via wires or wireless. In some instances, the sensors and the processor are electrically connected via wires.

The wearable devices can include an output device, where the output device and the processor are in electrical communication. In some instances, the processor and the output device are in wireless electrical communication such as Bluetooth. In some instances, the processor can compare actual ventilation rates to predicted values, from which ventilation rate above predicted (VRAP) can be calculated. Typically, this data is displayed on an output device, providing an instantaneous measure of stress for the interested party (e.g. user, investigator, or healthcare professional, etc.). Optionally, the data are recorded and stored. The apparatus is constructed to provide information about a subject's stress state.

In an exemplary system, the apparatus for measuring ventilation rate measures changing lung volumes via a thoracic strap around the chest equipped with an elastic section, one side of which is a magnet and the other side of which is a magnetic field sensor. Expansion and contraction of the thorax leads to varying distances between the magnet and sensor, which then be manifest as a change in signal strength, serving as a clear signal for respiration rates. With calibration, the sensor can be used to determine respiration volumes, as well. The activity sensor is also attached to the thoracic strap and measures activity via, for example, an accelerometer system, using an acceleration-based metric, such as vectorial dynamic body acceleration. The two signals (for ventilation rate and activity) are typically considered together to determine stress level in a human or in an animal.

a. Sensors

i. Breathing Rate Sensor

The device contains a sensor to measure ventilation rates (e.g. breaths per minute). The terms breathing rate and ventilation rate are used interchangeably herein. The ventilation sensor can be any suitable device, such as simple stress gauge, an accelerometer, or a magnet driven magnetometer-based system. In some instance, the magnet driven magnetometer-based system includes a magnet and a magnetometer.

In some instances, the ventilation sensor detects changes in (or displacements of the anteroposterior diameters of a subject's rib cage and/or abdomen, and/or axial displacement of the subject's chest wall when the ventilation sensors are disposed at selective anatomical positions. In some instances, the ventilation sensor is a magnet driven magnetometer-based system, which includes a pair of magnet and magnetic field detector, e.g. a magnetometer, where the pair is responsive to changes in spaced distances there between. For example, a strap and/or belt suitable for placing around a subject's chest and/or abdomen, which contains an elastic section, with a magnet attached to one end of the elastic section and a magnetic field detector, e.g. a magnetometer, attached to the other end. Expansion and contraction of the thorax lead to varying distances between the magnet and the detector, which manifest as a change in signal strength, serving as a signal for respiration rates. With proper calibration, these signals can be used to determine respiration volumes. The ventilation sensor can be used to measure changing lung volumes over time when attached around a subject's chest. In some instances, wireless sensors with the capability of measuring time delay in a signal sent from one sensor to another and thereby determine the distance between the two sensors can be used to measure the ventilation rate.

The ventilation sensors can be positioned at any appropriate position on a subject's body. Typically, the ventilation sensors are positioned at a subject's body part that doesn't move. In some instances, the ventilation sensors are positioned on a subject's torso. In some instances, the ventilation sensors are positioned on the chest of the subject. In some instances, the ventilation sensors are positioned at the abdomen of the subject.

The ventilation sensors can be in direct contact with the skin of a subject or indirect contact with the skin of the subject. In some instances, the ventilation sensors are fixed directly to the skin of the subject by any suitable means, e.g. by applying a tape or glue. In some instances, the ventilation sensors are fixed on a monitoring garment that can be tightly conformed to the body of the subject.

ii. Activity Sensor

The apparatus also contains an activity sensor, which can be any suitable devices that sense and transmit the body posture-motion signal, such as resting, walking, running, etc. In some instances, the activity sensor can be an accelerometer that records a subject's motion along one or more axes. The accelerometer measures proper acceleration, i.e., the acceleration (or rate of change of velocity) of a body in its own instantaneous rest frame. This differs from coordinate acceleration, being the acceleration in a fixed coordinate. Preferably, the activity sensor is a tri-axial accelerometer, which provides simultaneous measurements in three orthogonal directions, for analysis of all of the movements experienced by a subject. Each unit incorporates three separate sensing elements that are oriented at right angles with respect to each other. The tri-axial accelerometer can be used to measure the dynamism in a body's acceleration to provide the body movement metric in the analysis described herein.

The activity sensors can be positioned at any appropriate position on a subject's body, e.g. chest, abdomen, wrist, arm, etc. In some instances, the activity sensors can be at the same position as the ventilation sensors or at different positions as the ventilation sensors. In some instances, both the activity sensors and ventilation sensors are positioned on the chest of the subject. In some instances, both the activity sensors and ventilation sensors are positioned on the abdomen of the subject. In some instances, the activity sensors are positioned on the abdomen of the subject and the ventilation sensors are positioned on the chest of the subject. In some instance, the activity sensors are positioned on the chest of the subject and the ventilation sensors are positioned on the abdomen of the subject. In some instances, the activity sensors are positioned on a moving part of the body, e.g. wrist and/arm and the ventilation sensors are positioned on a body part that does not move, e.g. chest and/or abdomen.

The activity sensors can be in direct contact with the skin of a subject or indirect contact with the skin of the subject. In some instances, the activity sensors are fixed directly to the skin of the subject by any suitable means, e.g. by applying a tape or glue. In some instances, the activity sensors are fixed on a monitoring garment that can be tightly conformed to the body of the subject. For example, the activity sensors can be fixed on a band that is suitable to be worn on the wrist and/or arm of the subject or attached to a strap suitable to be worn around the chest of the subject and measure activity, e.g., using an acceleration-based metric (such as vectorial dynamic body acceleration) as a direct proxy for activity.

b. Support Structure

The wearable device is generally suitable to be directly attached to a subject or embedded in or carried by a support structure, such as a monitoring garment that is suitable to be worn by the subject. The monitoring garment is configured and adapted to cooperate with the wearable device and tightly conform to the body or a part of the body of a subject when secured thereon. The monitoring garment can include one or more sensors, e.g. ventilation sensor, activity sensors, and optionally additional sensors to monitor one or more additional physiological parameters, such as electrocardiogram, temperature, blood oxygen level, heart rate, pulse rate, blood pressure, etc. The monitoring garment can further include processors that are in electrical connection with the sensors.

The monitoring garment can include various garments or items that are adapted to tightly conform to at least a portion of a subject's body, such as a scuba wet suit, shirt, vest, jacket, band, strap, belt, and the like. In some instances, the monitoring garment is a thoracic strap around the subject's chest, a belt around the subject's abdomen (e.g. a belt), or combinations thereof. The sensors can be attached to different items of the monitoring garment, e.g. ventilation sensors attached to a strap around the chest and activity sensors attached to a belt around the abdomen of the subject. The sensors can also be attached to the same item of the monitoring garment, e.g. ventilation sensors and activity sensors attached to the same strap around the chest of the subject.

For example, ventilation sensors and activity sensors can be worn via a thoracic strap around the chest equipped with an elastic section, one side of which is a magnet and the other side of which is a magnetic field detector. Expansion and contraction of the thorax leads to varying distances between the magnet and detector, which then be manifest as a change in signal strength, serving as a clear signal for respiration rates. The activity sensors attached to the same thoracic strap measure activity via, for example, an accelerometer system, using an acceleration-based metric, such as vectorial dynamic body acceleration. Monitoring garment can be made with materials that are suitable for a wearable garment or clothing tightly conformed to the body or a body part of the subject, e.g. elastic materials. Exemplary elastic materials for the monitoring garment include, but are not limited to, polyurethane-polyurea copolymer such as LYCRA®, neoprene rubber, natural polyisoprene, synthetic polyisoprene, polybutadiene, chloroprene, butyl rubber, styrene-butadiene rubber, nitrile rubber, ethylene propylene rubber, epichlorohydrin rubber, polyacrylic rubber, silicone rubber, fluorosilicone rubber, fluoroelastomers, perfluoroelastomers, polyether block amides, chlorosulfonated polyethylene, ethylene-vinyl acetate, thermoplastic elastomers, proteins resilin and elastin, polysulfide rubber, and elastolefin.

In some instances, the monitoring garment can include additional materials, e.g. leather, synthetic leather, cotton, Velcro, and combinations thereof. For example, a circum-thoracic cotton and Velcro strap where the materials between the ventilation sensors are elastic.

c. Processor

A processor is commercially available. The processor performs mathematical analysis using an appropriate algorithm or signal processing on the data information provided by the sensors and calculates the ventilation rate and processes the acceleration data. Additionally, the processor can compare actual ventilation rates to predicted values, from which ventilation rate above predicted (VRAP) can be calculated. In some instances, the processor can also store data.

In some instances, the processor is a microprocessor board which can be easily integrated in the sensors. For example, the processor can be integrated in the magnetic field detector of a magnet driven magnetometer-based system or an accelerometer. In some instances, the processor can be embedded in or fixed on a support structure, e.g. a monitoring garment and electrically connected to the sensors. For example, the processor is embedded in or fixed on a thoracic strap and connected to the sensors by wires.

In some instances, the processor can transmit signal or data to an output device by a wireless transmitter that is commercially available. In some instances, the processer can be detached from the sensors and transfer data to an output device such as a computer.

d. Output Device

The output(s) from the sensors and calculations can be transmitted to an output device and displayed on the output device. Suitable output devices include a computer, watch, smart phone, personal digital assistant, exercise equipment, etc.

In some instances, the output device is portable and powered by a power source. Preferably the power source is a disposable or rechargeable battery.

The output device and the processor are in electrical communication, preferably are in wireless electrical communication. In some instances, the output device has a short-range wireless transceiver which is preferably a transmitter operating on a wireless protocol, e.g. Bluetooth, part-15, or 802.11. “Part-15” refers to a conventional low-power, short-range wireless protocol, such as that used in cordless telephones. The short-range wireless transmitter, e.g., a BLUETOOTH transmitter, receives information from the microprocessor and transmits this information in the form of a packet through an antenna. An external laptop computer or hand-held device features a similar antenna coupled to a matched wireless, short-range receiver that receives the packet. In some instances, the hand held device is a cellular telephone with a Bluetooth circuit integrated directly into a chipset used in the cellular telephone. In this case, the cellular telephone may include a Software application that receives, processes, and displays the information. In some instances, the wireless component may be a long-range wireless transmitter that transmits information over a terrestrial, satellite, or 802.11-based wireless network. Suitable networks include those operating at least one of the following protocols: CDMA, GSM, GPRS, Mobitex, DataTac. iDEN, and analogs and derivatives thereof. Alternatively, the handheld device is a pager or PDA.

### III. Methods of Using

The apparatus and methods described herein can be used with humans and wild animals, restrained or free-living. The apparatus can be used to detect stress in humans of interest because of medical condition or for sport and athletes, as well as for experiments in humans or animals that breathe air, such as mammals and birds, in physiology, psychology and other applications.

The method for determining a stress level in a subject typically includes: (a) measuring the ventilation rate over time of the subject; (b) measuring the activity over time of the subject, where steps (a) and (b) occur simultaneously; and (c) comparing the measured ventilation rate to a value predicted by a minimum ventilation rate to determine if the measured ventilation rate is greater than the predicted minimum ventilation rate for the activity level. The predicted ventilation rate for the activity level can be determined by comparing the ventilation rate with activity over time in the absence of a stress stimulus for a given activity level such as resting, walking, running, etc. The measurement in step (a) can be obtained using a ventilation rate measuring device, optionally the ventilation rate measuring device is a magnet driven magnetometer-based system, an accelerometer, or a stress gauge, preferably the ventilation rate measuring device is a magnet driven magnetometer-based system. The measurement in step (b) can be obtained using an activity measuring device, preferably the activity measuring device is an accelerometer, preferably a tri-axial accelerometer. The data from step (b) can be processed to determine the dynamic body acceleration (DBA). In some instance, the data from step (b) is processed to determine the vectorial sum of the dynamic body acceleration (VeDBA). The subject can be humans or animals that breathe air. In some instance, the method further includes (d) displaying the result from step (c) on an output device. In some instances, when the measured ventilation rate is greater than that predicted by the minimum ventilation rate for the same activity level, the subject is determined to be experiencing a stress response. In some instances, the measured ventilation rate is greater than that predicted by the minimum ventilation rate for the same activity level via changing the environmental conditions that the subject is exposed to.

For example, the apparatus for measuring ventilation rate measures changing lung volumes via a thoracic strap around the chest equipped with an elastic section, one side of which is a magnet and the other side of which is a magnetic field sensor. Expansion and contraction of the thorax leads to varying distances between the magnet and sensor, which then be manifest as a change in signal strength, serving as a clear signal for respiration rates. With calibration, the sensor can be used to determine respiration volumes, as well. The activity sensor is also attached to the thoracic strap and measures activity via, for example, an accelerometer system, using an acceleration-based metric, such as vectorial dynamic body acceleration.

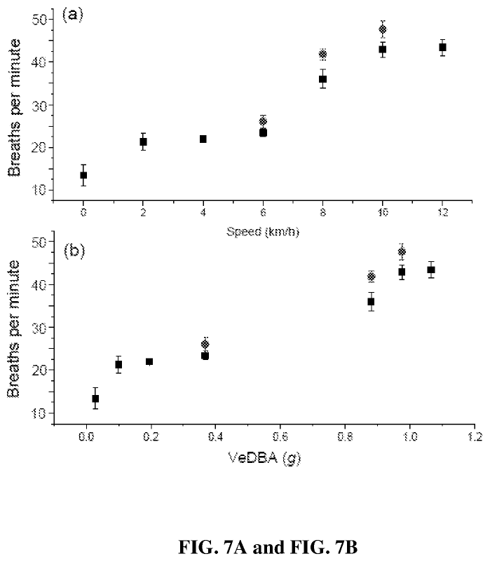

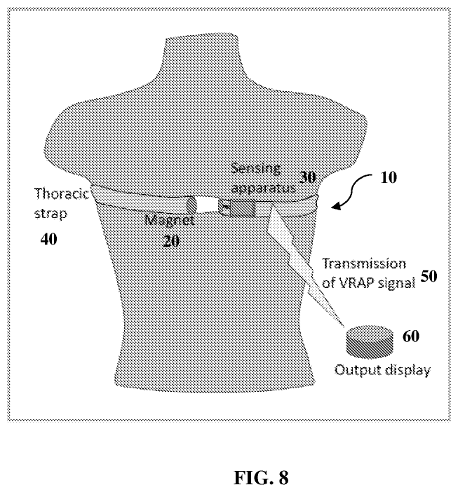

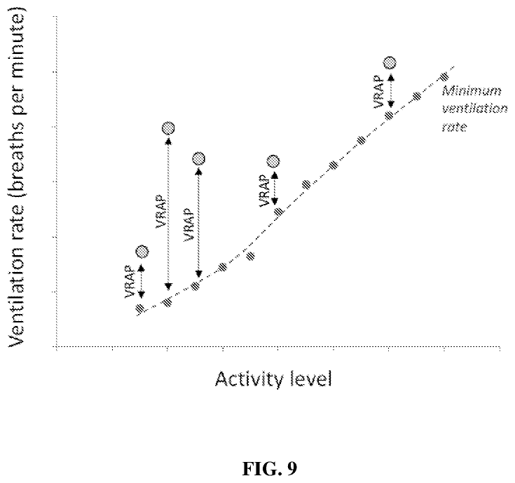

The two signals (for ventilation rate and activity) are typically considered together to determine stress level in a human or in an animal Activities over a period of time against ventilation rates derived from the activity sensors and ventilation sensors show a minimal ventilation rate corresponding to each level of activity, representing the non-stressed state. This minimum ventilation rate increases with the increase of activity level (FIG. 9). When a subject is stressed, the ventilation rate is higher than that predicted by the minimum ventilation rate (ventilation rate above predicted, “VRAP”). The extent of the difference between VRAP and the minimum ventilation rate line is a measure of the extent of the stress (FIGS. 7A and 7B).

In an exemplary measurement, a subject can wear the apparatus for a period of time, during which the apparatus would derive the minimum ventilation rate line from the lowest ventilation points against activity. The VRAP can be calculated in real time or the data measured can be recorded and stored and the VRAP can be calculated after the measurement period. In some instances, the VRAP can be calculated in real time and displayed on an output device, e.g. a watch or smart phone, providing an instantaneous measure of stress. The subject can wear the apparatus for a period between about 1 hour and about 1 year, between about 1 hour and about 6 months, between about 1 hour and about 5 months, between about 1 hour and about 4 months, between about 1 hour and about 3 months, between about 1 hour and about 2 months, between about 1 hour and about 1 month, between about 1 hour and about 25 days, between about 1 hour and about 20 days, between about 1 hour and about 15 days, between about 1 hour and about 10 days, between about 1 hour and about 6 days, between about 1 hour and about 5 days, between about 1 hour and about 4 days, between about 1 hour and about 3 days, between about 1 hour and about 2 days, between about 1 hour and about 1 day, between about 1 day and about 6 days, between about 1 day and about 7 days, between about 1 day and about 8 days, between about 1 day and about 9 days, between about 1 day and about 10 days, between about 1 day and about 30 days, between about 1 day and about 60 days, between about 6 day and about 6 months, and between about 6 day and about 1 year.

a. Acceleration-Based Metrics

The calculation of dynamic body acceleration (DBA) and posture can be used to specifically and generally quantify activity in a human or animal (Shepard, et al., Endangered Species Research, 10:47-60 (2008); Wilson, et al., Journal of Animal Ecology, 75:1081-1090 (2006)).

Suitable methods for calculating animal posture for an animal that is tagged with an tag including a tri-axial accelerometer therein include smoothing the acceleration channels from an animal-attached tag to provide the “static” acceleration, i.e. the value that is nominally due to the earth's gravitational field (Shepard, et al., Aquatic Biology, 4:235-241 (2008); Shepard, et al., Endangered Species Research, 10:47-60 (2008)). The static element of any acceleration (A) data point i (SA_{c},i) is given by:

$${S}_{i} = \frac{1}{w} {\sum }_{j = i - \frac{w}{2}}^{i + \frac{w}{2}} {A}_{j} \qquad Equation ( 1 )$$

where w is the smoothing window. The sine or cosine of this can be used to determine the angle of the tag with respect to gravity (pitch and roll) and thereby the posture of the animal (Wilson, et al., Movement Ecology, 4:22 (2016)).

In order to derive the dynamic body acceleration per orthogonal axis channel (DA_{c}), the smoothed values (SA_{c}) are subtracted from the raw values per channel (A_{c}) following:

DA_{c}=A_{c}−SA_{c} Equation (2)

All three dimensional axes of acceleration are added to provide a summed acceleration metric. This sum can be the vectorial sum of the dynamic body acceleration (VeDBA), which uses the dynamic components of acceleration (DAₓ, DA_{y}, and DA_{z}) to take the vectorial length of the dynamic acceleration vector (Qasem, et al., PLoS One, 7:e31187 (2012)) via:

VeDBA=\sqrt{DAₓ²+DA_{y}²+DA_{z}²} Equation (3)

b. Ventilation-Based Metrics

A minimum, predicted ventilation rate (breaths per minute) can be determined by reviewing a person's ventilation rate when engaged in various activities over time and determining a minimum ventilation rate corresponding to each level of activity. Increasing activity, results in an increase minimum ventilation rate. This represents the non-stressed state.

Ventilation rate above predicted (VRAP) refers to a measured ventilation rate that is higher than the minimum (predicted) ventilation rate for a given activity level. As shown in FIG. 9 and FIGS. 7A-7B, difference between the measured ventilation rate and the minimum ventilation rate is the VRAP. When person (or animal) is stressed, the ventilation rate is higher than that predicted by the minimum ventilation rate line (ventilation rate above predicted (VRAP)). The extent of the difference between VRAP and the minimum ventilation rate line is a measure of the extent of the stressor (FIG. 9 and FIGS. 7A-7B).

The two signals (for ventilation rate and activity) are considered simultaneously. Stress is determined by comparing actual ventilation rates to predicted values, from which ventilation rate above predicted can be calculated, and displayed on an output device as required by the user, providing an instantaneous measure of stress for the interested party. Optionally, the data are recorded and stored.

A subject can wear the apparatus for a period of time, during which time the system derives the minimum ventilation rate line from the lowest ventilation points against activity. Then, in real time, the system calculates VRAP at each time point, and optionally displays the data on an output device (such as a watch or smart phone) as required by the user. Optionally, the data is recorded. This method provides an instantaneous measure of stress for the interested party (FIG. 9).

The subject can wear the apparatus to monitor his/her stress levels and modify his/her behavior or exposures to one or more stress stimuli, to decrease future stress levels. Similarly, a medical practitioner can monitor the data and use it to identify one or more stress stimuli, and optionally, prescribe modifications to the subject's routines to minimize or prevent exposures to one or more stress stimuli.

The apparatus and methods described herein can be used with animals that breathe air, such as mammals and birds, whether in a scientific study, such as to ensure that procedures for dealing with the animals have minimum impact on the study animals, or for diagnostic purposes to evaluate the animal's breathing and/or activity. The apparatus and methods can also be used as a lie detector to evaluate the physiological responses of a person being questioned.

The present invention will be further understood by reference to the following non-limiting examples.

### EXAMPLES

### Example 1. Changes in Behavior and State Over Time Manifest Via Acceleration-Based Metrics in an Animal

Materials and Methods

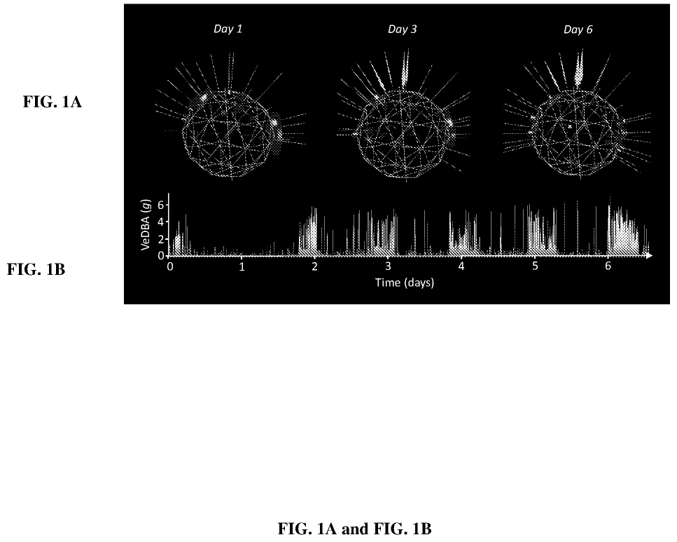

General animal activity was measured for a European badger Meles tagged in northern Ireland over 6 full days using dynamic body acceleration (DBA) alone. FIG. 1B shows the VeDBA of the badger and the gradual increase in movement dynamism following initial capture, sedation with ketamine, and release.

The same data can be interrogated by using visualisations that incorporate both the static and the dynamic accelerations. A tri-axial plot of the static acceleration tends to have the data all lie on the surface of a sphere that has a radius of 1 g, with the position on the surface being dependent on the animal body posture. The nominal limits (in g) to the co-ordinates of this sphere (following a y-, x- and z-axis system) are: 1, 0, 0 (North Pole); −1, 0, 0 (South Pole); and 0, 0, 1; 0, 0, −1; 0, 1, 0; and 0, −1, 0 for the 4 equatorial limits. This ‘g-sphere’ representation (Wilson et al. 2016) allows all postures to be visualised intuitively within one plot, with some idea of the representation of time to posture. However, much data in one locality hide other data in this locality so the visualisation cannot represent, in this format, large quantities of data, and nor does it incorporate the dynamic component of acceleration.

Both these problems are addressed by dividing the surface of the g-sphere into facets (for details see Wilson et al. 2016) and the number of data points within each facet summed. The VeDBA values of all points within any given facet are then classified into frequency bins. The distribution of the VeDBA frequencies per facet is then represented by discs on a spine emanating from the centre of the facet and projecting into space. The width of the discs represents the width of the VeDBA bin while the diameter represents the proportion of the data residing in any given bin. The process is conducted for all facets of the sphere. This ‘urchin plot’ gives an immediate picture of time and dynamism allocated to posture, highlighting modes which may constitute particular behaviours (FIGS. 1A and 1B).

Results

Results are shown in FIGS. 1A and 1B. FIG. 1A shows spherical plots representing body angle by spine position (spines on the North pole show periods when the animal was horizontal, with increasing proximity to the equator showing the animal lying more completely on its side). Increased activity allocated to body angle is shown by discs on the spines, with disc thickness indicating VeDBA bins (starting from low VeDBA values close to the disc surface to increasing VeDBA values farther away from the sphere) while disc diameter indicates time at VeDBA and posture. The extended times spent essentially immobile (body postures indicate most likely sleeping) and the slow return to higher VeDBA values at the North pole of the sphere indicate that the animal was moving about on all fours.

In the case of data acquired from a badger Meles meles (FIGS. 1A and 1B), the urchin plots illustrate both variation of time and energy allocated to various postures over the 6 days the animal was monitored following capture, sedation and release. The urchin visualisation shows how the first days involved much sleeping in positions for long periods (without changing position) with little normal locomotion, slowly giving way to normal activities. Closer inspection of the urchin provides more specific data. The technology provides valuable quantification of activity.

### Example 2. Changes in Behavior and State Over Time Manifest Via Acceleration-Based Metrics in Humans

A. Effects of Emotion on Activity

The use of urchin plots for determining activity in humans was investigated by in 20 human participants. Each participant was equipped with a back-mounted accelerometer and asked to watch two 4-minute films, one which was considered to elicit feelings of happiness (a clip of two people drinking beer supposedly laced with helium, which gave them high pitched voices), while the other was chosen to elicit sadness (a death scene from Disney's ‘Bambi’). Following each film, participants were asked to walk down an empty 40 m corridor to rate their reaction to the films on a piece of paper. Their data sets were then processed by being allocated to urchin plots, within which facet dispersion (320 facets were used to cover the g-sphere) and VeDBA allocation to facet were examined.

This showed a significant effect of film type (Happy or Sad) (χ²_{(2,1)}=17.99, p<0.0001), time spent walking (χ²_{(1,1)}=6.69, p=0.009), and facet dispersion (χ²_{(1,1)}=6.54, p=0.011), on mean (log) VeDBA. The data from walks of a male participant after having watched a happy and a sad film clips was analyzed. There was a decrease in the number of facets ‘occupied’ by the acceleration data in the ‘sad’ condition, which indicated reduced ‘facet dispersion’ manifesting reduced ‘swagger’.

B. Measurement of Micro-Movements in Humans

State effects manifest in movement patterns may also be observable in micro-movements of the body's external surface (Flavel et al. 2012). As part of a study to investigate the effects of nicotine, tri-axial accelerometers sampling at 800 Hz were attached to the fingernails of 20 smokers and looked at the manifestation of tremor prior to, and during, smoking. Analysis of the accelerometer outputs using a fast Fourier transformation showed a marked increase in the intensity (signal strength) of tremors across all individuals, demonstrating that the change in chemical state was readily detected using the accelerometry approach.

### Example 3. Changes in Physiology Manifest Via Magnetometry-Based Metrics in Humans and Animals (Penguins)

Materials and Methods

Ventilation Rates

A custom magnet-driven magnetometer (MDM) was constructed, consisting of a tri-axial magnetometer (Honeywell HMC5883L, supplied by Wildbyte Technologies Ltd, Singleton Park, Swansea, UK), which recorded (at 40 Hz) the proximity of a distant neodymium boron magnet (10×7 mm dia) via changes in magnetic field intensity. The basic principal of functioning relied on having a circum-thoracic strap containing an inbuilt system whereby the magnet-sensor distance varied with expansion of the rib change and thereby with lung volume. For this, both magnetometer (encased in a tightly fitted plastic case) and magnet were placed ca. 5 cm apart, on a circum-thoracic cotton and velcro strap except for the material between magnet and magnetometer, which was made of elastic.

The relationship between magnetic signal and inspirational volume and breath frequency was ascertained by asking 8 participants lying in three different positions (on both sides and on their back) wearing the magnet-driven magnetometer to breathe into a face mask connected to a VYNTUS® IOS (CareFusion 303, Inc., San Diego, Calif.), a device with a clinically proven lung function testing device frequently used in medicine for determining tidal volume and breathing rates with high accuracy. Breathing into the face mask connected to a VYNTUS® IOS (CareFusion 303, Inc., San Diego, Calif.) was used to confirm that all breaths represented by the magnet-driven system corresponded to real breaths.

The same magnet-driven magnetometer system was tested on wild animals. The same magnet-driven magnetometer system was attached to 8 wild Magellanic penguins Spheniscus magellanicus held in darkened boxes and their ventilation rates were measured.

Results

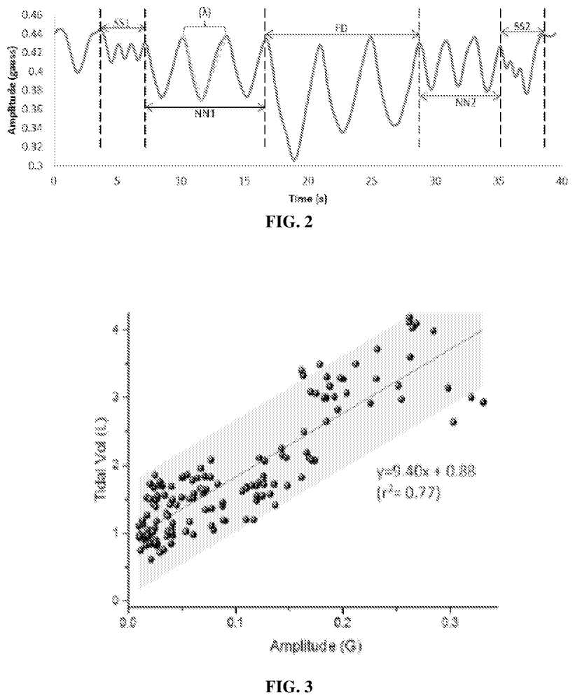

The system showed breathing patterns extremely clearly with obvious time and amplitude differences between breathing regimes according to different tidal volumes and respiration rates (FIG. 2).

FIG. 2 shows an example of how a magnet-driven magnetometer on an elasticated circum-thorax strap was used to determine ventilation rates in humans. The trace shows the raw signal in relation the breathing. For this exercise, the participant was asked to lie and then execute three types of breathing patterns; SS1 & SS2—Short, sharp breaths, NN1 & NN2—Normal breaths, and FD—Full, deep breaths. λ indicates the wavelength (and hence the ventilation rate).

Peak-to-peak analysis of data was successful in identifying all 654 of 654 breaths executed when magnetometer signals were compared to the breath-by-breath data of the VYNTUS® IOS.

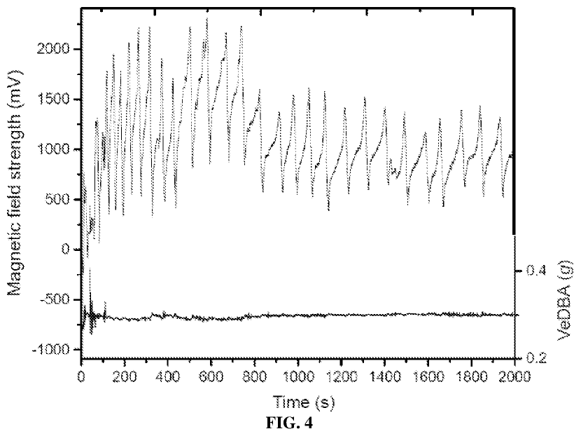

Individual correlations between tidal volume and amplitude were very clear for all participants (FIG. 3) as well as between participants and positions, including all positions and participants combined, and were highly significant (p<0.001) (Table 1) although variation is high.

FIG. 3 demonstrates that the amplitude in the breathing signal from a magnet-driven magnetometer on an elasticated circum-thorax strap may be used to determine tidal volume (ascertained by independent means) in humans across body positions.

Specifically, use of ANOVAs to compare GLMs with and without terms (positions and participants) showed that tidal volume had a significant effect on amplitude (Estimate=6.26, Std Error=0.15, t=42, p<0.001) and that body position (1=On back, 2=on right side, 3=on left side) also had a significant interaction with volume and its effect on amplitude (χ²(2,1304)=81.23, p<0.001).

Table 1. Best fit linear relationships (y=mx+c) between y (the magnetometer signal amplitude (gauss)) and x (the tidal volume (L) for participants lying in three different positions (1=on back, 2=on right side, 3=on left side). The SEM² is the standard error of the mean and * shows statistical significance at the shown level.

TABLE 1

[symbol]

| Participant | Position | Gradient | Intercept | r² | P-value | SEM² |
|---|---|---|---|---|---|---|
| 1 | 1 | 13.16 | 0.33 | 0.96 | <0.001* | 0.21 |
| [symbol] | 2 | 12.15 | 0.99 | 0.90 | <0.001* | 0.11 |
| | 3 | 9.17 | 0.77 | 0.79 | <0.001* | 0.09 |
| | All | 9.40 | 0.89 | 0.77 | <0.001* | 0.07 |
| 2 | 1 | 33.31 | 0.26 | 0.91 | <0.001* | 0.09 |
| [symbol] | 2 | 35.46 | 0.24 | 0.89 | <0.001* | 0.08 |
| | 3 | 29.17 | 0.34 | 0.90 | <0.001* | 0.07 |
| | All | 32.76 | 0.28 | 0.89 | <0.001* | 0.05 |
| 3 | 1 | 9.16 | 0.60 | 0.92 | <0.001* | 0.16 |
| [symbol] | 2 | 6.85 | 0.61 | 0.87 | <0.001* | 0.14 |
| | 3 | 5.13 | 0.56 | 0.89 | <0.001* | 0.14 |
| | All | 6.09 | 0.68 | 0.80 | <0.001* | 0.10 |
| 4 | 1 | 14.61 | 0.03 | 0.95 | <0.001* | 0.08 |
| | 2 | 14.74 | 0.11 | 0.96 | <0.001* | 0.06 |
| | 3 | 13.82 | 0.14 | 0.92 | <0.001* | 0.05 |
| | All | 13.97 | 0.09 | 0.93 | <0.001* | 0.04 |
| 5 | 1 | 22.16 | 0.00 | 0.96 | <0.001* | 0.14 |
| | 2 | 11.15 | 0.01 | 0.93 | <0.001* | 0.09 |
| | 3 | 6.37 | 0.24 | 0.96 | <0.001* | 0.13 |
| | All | 13.63 | 0.12 | 0.61 | <0.001* | 0.07 |
| 6 | 1 | 26.03 | 0.39 | 0.93 | <0.001* | 0.13 |
| | 2 | 14.66 | 0.40 | 0.72 | <0.001* | 0.09 |
| | 3 | 9.62 | 0.42 | 0.91 | <0.001* | 0.10 |
| | All | 12.28 | 0.54 | 0.64 | <0.001* | 0.06 |
| 7 | 1 | 9.35 | 0.38 | 0.91 | <0.001* | 0.10 |
| | 2 | 8.49 | 0.25 | 0.94 | <0.001* | 0.11 |
| | 3 | 4.96 | 0.31 | 0.85 | <0.001* | 0.08 |
| | All | 5.76 | 0.44 | 0.70 | <0.001* | 0.06 |
| 8 | 1 | 15.41 | 0.12 | 0.94 | <0.001* | 0.27 |
| | 2 | 5.69 | 0.31 | 0.87 | <0.001* | 0.12 |
| | 3 | 5.01 | 0.16 | 0.83 | <0.001* | 0.13 |
| | All | 5.35 | 0.40 | 0.62 | <0.001* | 0.09 |
| ALL | P1 | 11.42 | 0.50 | 0.75 | <0.001* | 0.004 |
| ALL | P2 | 6.65 | 0.63 | 0.60 | <0.001* | 0.005 |
| ALL | P3 | 4.96 | 0.50 | 0.75 | <0.001* | 0.006 |
| ALL | ALL | 6.26 | 0.65 | 0.57 | <0.001* | 0.003 |

[symbol]

indicates data missing or illegible when filed

Deployment of the same magnet-driven magnetometer system on the 8 wild Magellanic penguins Spheniscus magellanicus held in darkened boxes showed that breathing patterns were reflected very well in magnetometer signal amplitude so that breathing rate could readily be determined according to condition (FIG. 4). As shown in FIG. 4, the respiration rates could be examined with respect to activity, as manifest by VeDBA. Under these conditions, in all 8 penguins the respiration rate was not simply dependent on activity with, for example, appreciable variation in respiration rate even during constant conditions of exercise (FIG. 4), indicating that ‘stress’ is an important element modulating ventilation rates.

### Example 4. Changes in Physiology Manifest Via Movement-Corrected Ventilation Metrics

Materials and Methods

A combined tri-axial accelerometer in tandem with a system for determining breathing rate (Zephyr bio-harness) was tested on one human while undertaking various tasks (sitting, standing, walking) and being submitted to different stimuli (exposure to calm music, loud music, and horror films).

Results

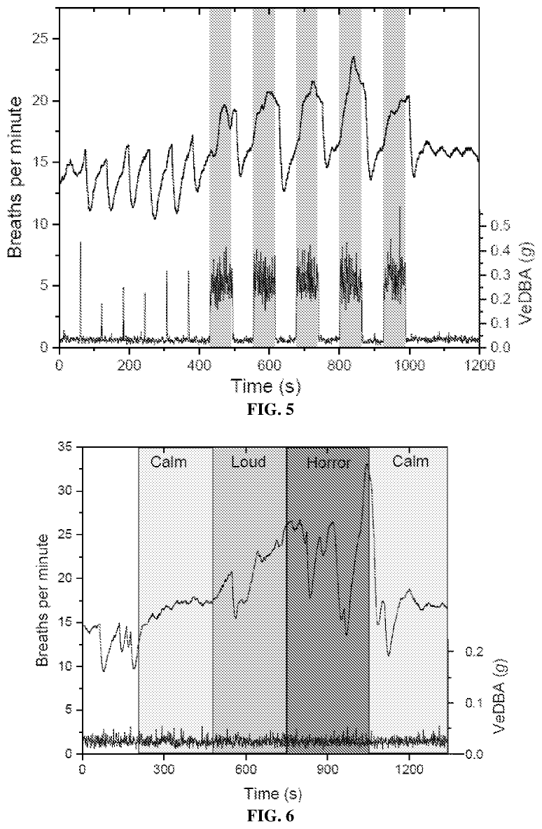

This pilot work showed that breath rates for particular activities preceded those activities in an anticipatory manner (FIG. 5) both in terms of increases in breathing rates preceding higher activities and decreases in breathing rates preceding lower activities. Anticipation of activity therefore appears to be enough to change ventilation rates.

Beyond that, for particular activities (all of which have defined VeDBAs alluding to metabolic rate (Halsey et al. 2011), and thus ventilation rate), breathing rate and variance changed according to perceived stress. For example, in a sitting, immobile subject, changes in ventilation rates associated with different environmental stimuli were observed (FIG. 6). In FIG. 6, the VeDBA is shown for the same period, which demonstrates that the changes in breathing rate are not a consequence of activity. For comparison, the mean VeDBA value for someone walking with an upper back-mounted accelerometer is approximately 0.35 g (cf. FIGS. 7A and 7B).

This effect was not just manifest during minimal exercise conditions because environmentally elicited breathing rates were additive to exercise-linked breathing rates (FIGS. 7A and 7B) irrespective of whether the metric for exercise was speed (see FIG. 7A) or the more general activity metric, VeDBA (see FIG. 7B). The breathing rates between the two conditions dissociate, with the ‘music’ conditions being consistently higher than ‘expected’.

### Example 5. Stress Sensor and Measuring the Stress Metric VRAP (Ventilation Rate Above Predicted)

Materials and Methods

As described above.

Results

The stress sensor used two principal elements:
(i) an apparatus for measuring ventilation rates; and
(ii) an apparatus that measures activity.

In a schematic, graphical representation of activity over an extended period against breathing rates derived from the use of these two apparatuses, there is apparent a minimal ventilation rate corresponding to each level of activity (representing the non-stressed state), with this minimum ventilation rate increasing with increasing activity (FIG. 9). This is the non-stressed state (represented by the dashed line in FIG. 9).

If, however, a person (or animal) is stressed, the ventilation rate is higher than that predicted by the minimum ventilation rate line (ventilation rate above predicted—VRAP). The extent of the difference between the actual ventilation rate and the minimum ventilation rate line is the VRAP, which is a measure of stress experienced by the individual or animal (see FIGS. 9, 7A and 7B).

### REFERENCES

Flavel, S. C., J. D. Koch, J. M. White, and G. Todd. 2012. Illicit stimulant use in humans is associated with a long-term increase in tremor. PloS one 7:e52025.
Halsey, L. G., E. L. Shepard, and R. P. Wilson. 2011. Assessing the development and application of the accelerometry technique for estimating energy expenditure. Comparative Biochemistry and Physiology Part A: Molecular & Integrative Physiology 158:305-314.
Qasem, L., A. Cardew, A. Wilson, I. Griffiths, L. G. Halsey, E. L. Shepard, A. C. Gleiss, and R. Wilson. 2012. Tri-axial dynamic acceleration as a proxy for animal energy expenditure; should we be summing values or calculating the vector? PLoS One 7:e31187.
Shepard, E. L., R. P. Wilson, L. G. Halsey, F. Quintana, A. G. Laich, A. C. Gleiss, N. Liebsch, A. E. Myers, and B. Norman. 2008a. Derivation of body motion via appropriate smoothing of acceleration data. Aquatic Biology 4:235-241.
Shepard, E. L., R. P. Wilson, F. Quintana, A. G. Laich, N. Liebsch, D. A. Albareda, L. G. Halsey, A. Gleiss, D. T. Morgan, and A. E. Myers. 2008b. Identification of animal movement patterns using tri-axial accelerometry. Endangered Species Research 10:47-60.
Wilson, R. P., M. D. Holton, J. S. Walker, E. L. Shepard, D. M. Scantlebury, V. L. Wilson, G. I. Wilson, B. Tysse, M. Gravenor, and J. Ciancio. 2016. A spherical-plot solution to linking acceleration metrics with animal performance, state, behaviour and lifestyle. Movement ecology 4:22.
Wilson, R. P., C. R. White, F. Quintana, L. G. Halsey, N. Liebsch, G. R. Martin, and P. J. Butler. 2006. Moving towards acceleration for estimates of activity-specific metabolic rate in free-living animals: the case of the cormorant. Journal of Animal Ecology 75:1081-1090.

1. A method for determining a stress level in a subject, comprising:
a. measuring the ventilation rate over time of the subject,
b. measuring the activity over time of the subject, wherein steps (a) and (b) occur simultaneously, and
c. comparing the measured ventilation rate to a value predicted by a minimum ventilation rate to determine if the measured ventilation rate is greater than the predicted minimum ventilation rate for the activity level.

2. The method of claim 1, wherein the predicted ventilation rate for the activity level is determined by comparing the ventilation rate with activity over time in the absence of a stress stimulus for a given activity level.

3. The method of claim 1, wherein the measurement in step (a) is obtained using a ventilation rate measuring device, optionally the ventilation rate measuring device is a magnet driven magnetometer-based system, an accelerometer, or a stress gauge, preferably the ventilation rate measuring device is a magnet driven magnetometer-based system.

4. The method of claim 1, wherein the measurement in step (b) is obtained using an activity measuring device, preferably the activity measuring device is an accelerometer, preferably a tri-axial accelerometer.

5. The method of claim 1, wherein the data from step (b) is processed to determine the dynamic body acceleration (DBA).

6. The method claim 1, wherein the data from step (b) is processed to determine the vectorial sum of the dynamic body acceleration (VeDBA).

7. The method of claim 1, wherein the subject is a human.

8. The method of claim 1, wherein the subject is an animal that breathes air.

9. The method of claim 1, further comprising (d) displaying the result from step (c) on an output device.

10. The method of claim 1, wherein when the measured ventilation rate is greater than that predicted by the minimum ventilation rate for the same activity level, the subject is determined to be experiencing a stress response.

11. The method of claim 1, wherein when the measured ventilation rate is greater than that predicted by the minimum ventilation rate for the same activity level, changing the environmental conditions that the subject is exposed to.

12. A wearable apparatus for measuring ventilation rate and activity level in a subject, comprising a ventilation rate sensor and an activity sensor, preferably wherein both sensors are on the same support structure.

13. The wearable apparatus of claim 12, wherein the support structure is a thoracic strap.

14. The wearable apparatus of claim 12, further comprising a processor for processing the data from the sensors.

15. The wearable apparatus of claim 14, wherein the processor and the sensors are in electrical communication, preferably are in electrical communication via wires.

16. The wearable apparatus of claim 14, further comprising an output device, wherein the output device and the processor are in electrical communication, preferably are in wireless electrical communication.

17. The wearable apparatus of claim 12, wherein the ventilation sensor comprises a magnet and a magnetometer.

18. The wearable apparatus of claim 12, wherein the activity sensor is an accelerometer, optionally a tri-axial accelerometer.

19. The wearable apparatus of claim 12 comprising more than one ventilation sensors and/or more than one activity sensors.
